(19) United States
(12) Patent Application Publication
(10) Pub. No.: US 2021/0386362 A1
(43) Pub. Date: Dec. 16, 2021

(54) CHILDBIRTH AND LABOR MONITORING METHOD AND SYSTEM

(71) Applicant: Fructus Design, LLC, McHenry, IL (US)

(72) Inventor: Slawomir WOJTYSIAK, McHenry, IL (US)

(21) Appl. No.: 17/360,512

(22) Filed: Jun. 28, 2021

Related U.S. Application Data
(63) Continuation-in-part of application No. 17/345,372, filed on Jun. 11, 2021.
(60) Provisional application No. 63/037,701, filed on Jun. 11, 2020.

(51) Int. Cl. A61B 5/00

(52) U.S. Cl. CPC A61B 5/4356; A61B 5/0022; A61B 5/6824; A61B 2562/0247; A61B 5/742

(57) ABSTRACT

A method and system for monitoring the childbirth and the laboring process included a device designed to assist pregnant women in the first stage of labor in the form of a device that fits in a woman's hand (or worn on the wrist) and has a trigger that can be pressed at the onset of a contraction, and released when the contraction ends. The trigger is pressure-sensitive and can help to track the contractions' intensity level simultaneously.

### CROSS REFERENCE TO RELATED APPLICATION

This is a continuation-in-part of U.S. patent application Ser. No. 17/345,372 filed on Jun. 11, 2021, which in turn claims priority to U.S. Provisional Patent Application Ser. No. 63/037,701 file Jun. 11, 2020, all herein incorporated by reference.

The present invention relates to a method, system and device for childbirth and labor and in particular a process and system for monitoring childbirth and the laboring process.

### BACKGROUND OF THE INVENTION

There are many methods and systems for monitoring the childbirth and laboring process. These include but are not limited to tracking a mother's contractions and noting the time between them. More recently, various tools or devices have been developed which assist in the recording of contractions in order to assist calculating times between contractions including smartphone apps.

### SUMMARY OF THE INVENTION

The present invention is directed to a unique method, system and device for monitoring the childbirth and the laboring process. Included is a unique device specifically designed to assist pregnant women prior to, and in the first stage of labor. Advantageously the device fits comfortably in a woman's hand, and has a small trigger that can be pressed at the onset of a contraction, and released when the contraction ends. The trigger is also pressure-sensitive or force sensitive, and can help to track the contractions' intensity level simultaneously. The timer also has a small screen that displays data in real time. The device has a wireless communication system for connecting the device to a remote device such as a smartphone running an app, cloud server/database or healthcare facility server. This gives laboring women a simple, straightforward option for keeping track of their contractions, and can also help their care providers (doctors, midwives, nurses, spouses, etc.) gain a better idea of how labor is progressing. This product eliminates the inconvenience of conventional “contraction timing” of some conventional smartphone apps that require fiddling with one's smartphone during the hard work of labor. Instead, this contraction timer is immediately at hand when needed. Other benefits include: caregiver dashboard, periodic care reminders, SOS button, logging of non-contraction events, call button, automatic SMS alerts.

The present invention in one form thereof is directed to a device for monitoring the childbirth and labor process. The device has a housing with a trigger. The trigger is pressure-sensitive or force sensitive. For example, as force is applied to the trigger the device identifies this as being indicative of contraction intensity.

In one further form thereof, the device records contraction data generated when pressure or force is applied to the trigger. In an alternative further form, the device has a wireless communication system for transmitting contraction data generating when pressure is applied to the trigger. The communication system may include communication by Bluetooth and/or Wi-Fi and/or cellular.

In one additional alternative form, the device may have a display for displaying contraction data generated as a result of depressing the trigger and pressure applied to the trigger.

In still another further form the wireless communication system is connected to a remote device such as a smartphone running an app, cloud server/database or healthcare facility server.

In still yet a further form, the trigger may have a locking feature that locks the trigger in place. For example, the locking feature may comprise a ratcheting mechanism that incrementally locks the trigger in place as the trigger is depressed from a fully extended position, natural or quiescent position to a fully depressed position.

In still yet another further alternative form, the trigger may have a damper to soften the return of the trigger after the trigger has been pressed from a partial or fully depressed position to its natural or quiescent position.

The present invention, in another form thereof is directed to an ambulatory device for measuring the frequency and duration of uterine contraction. The ambulatory device has a processor circuit, an analog-to-digital convertor coupled to the processing unit and a biased trigger sensor. The sensor is configured to receive a manually applied time-varying input force signal over a monitoring period. The trigger sensor is coupled to the analog-to-digital convertor.

The device also has a wireless transceiver coupled to the processor. The trigger sensor can output a time-varying analog signal to the analog-to-digital convertor. The analog signal is monotonically increased with an increasing manually applied input force signal and is representative of the manually applied input force signal over the monitoring. The processing circuit is adapted to transmit a digital output signal via the wireless transceiver. The digital output signal is indicative of the manually applied input force over the monitoring.

### BRIEF DESCRIPTION OF THE DRAWINGS

The present invention will now be described with regard to the figures as filed in which:

FIG. 1 is a side elevation view of a device for assisting a pregnant woman in childbirth and the laboring process in accordance with the present invention.

FIG. 2 is a top plan view of the device of FIG. 1.

FIG. 3 is a cross sectional exploded view of the device of FIG. 1.

FIG. 4 is a partial breakaway sectional view of an alternative device showing its ratcheting mechanism in accordance with the present disclosure.

FIG. 5 is an alternative device in accordance with the present disclosure.

FIG. 6 is the device of FIG. 5 shown from the other side.

FIG. 7 is yet another alternative device in accordance with the present disclosure.

FIG. 8 is the device of FIG. 7 shown from the other side.

FIG. 9 is a flowchart showing an exemplary system using the device and implementing the method in accordance with the present disclosure.

### DETAILED DESCRIPTION OF THE PREFERRED EMBODIMENTS

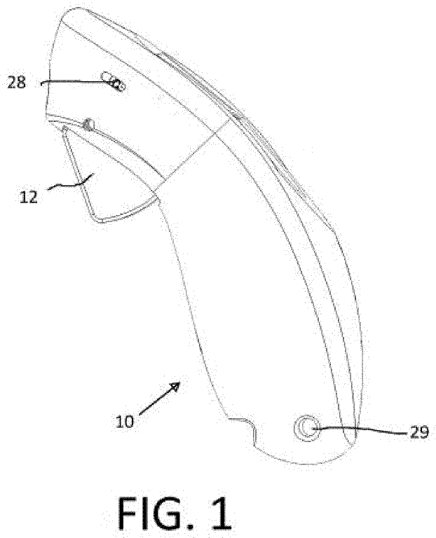

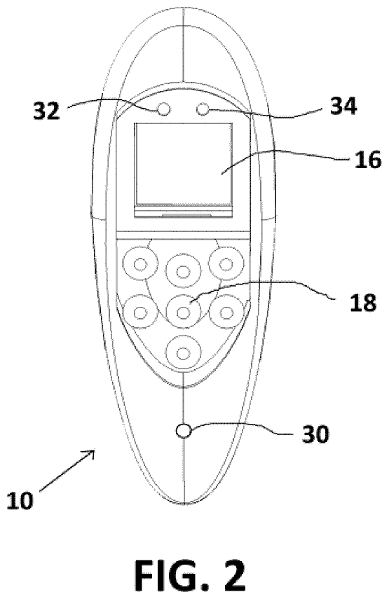

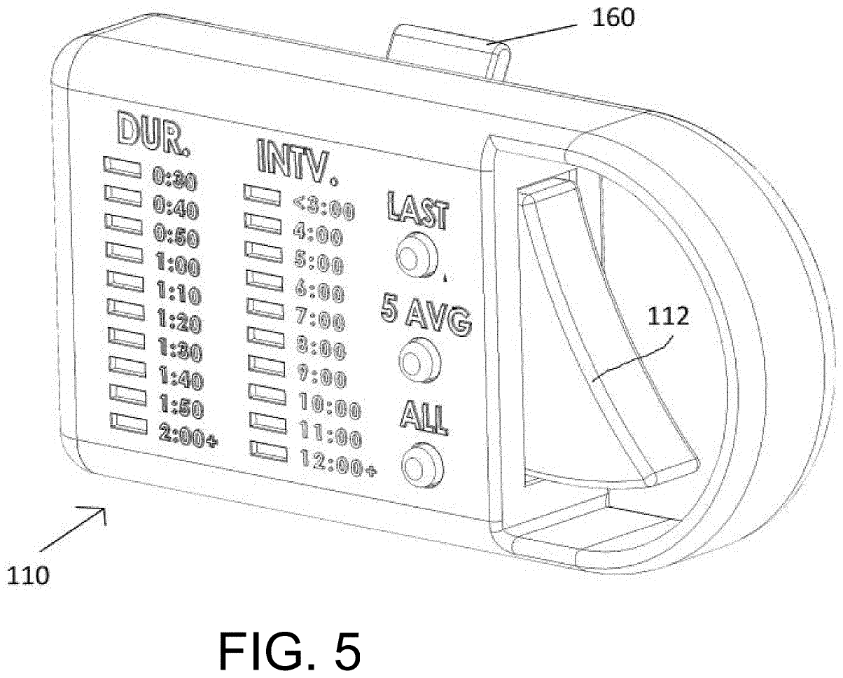

The present method and system advantageously includes a wireless battery operated device that is handheld and adapted to comfortably fit in the hand of an expectant mother. The device can have differing shapes and forms including the device of FIGS. 1-3, as well as the alternative smaller designs shown in the embodiments of FIGS. 5 and 6, and FIGS. 7 and 8, respectively.

Referring now specifically to the embodiment of FIGS. 1-3, device 10 has an index finger actuatable, spring-loaded trigger 12. The trigger 12 has a natural or quiescent position, fully extended and biased via a spring 14. Applying pressure to trigger 12 by ones index finger moves the trigger 12 from its natural state to a fully depressed state.

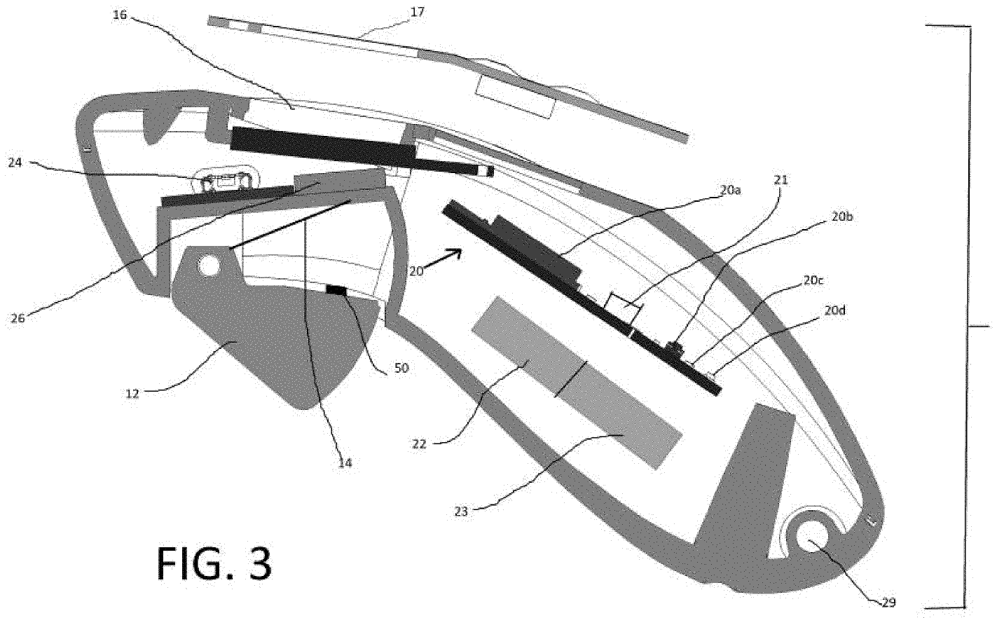

As best shown in the exploded, cross section of the device of FIG. 3, the device 10 has a display 16, transparent display cover 17, membrane keypad 18, main circuit board 20, main daughterboard 21, rechargeable battery 22, auxiliary rechargeable supplemental battery 23, charging and software maintenance USB port 24, and Hall Effect sensor(s) 26. As an alternative to membrane keypad 18, the device may have a joystick or other suitable means for input in accordance with this disclosure.

The trigger 12 may contain detents so less effort is required to hold the trigger 12 in a specific spot. Display 16 can show data relating to contractions and other parameters pertaining to the operation of device 10. These parameters include but are not limited to a wireless connection, battery level, etc.

Conveniently located in the bottom portion of device 10 is a hole 29 that allows one to attach a lanyard or other means for conveniently holding the device 10 at one side.

USB port 24 allows batteries of the device 10 to be charged using a conventional charging system known to those skilled in the art to charge main battery 22 and auxiliary battery 23. Alternatively, rather than having a rechargeable battery, the device 10 can be adapted for use with removable batteries such as AA or AAA batteries. In addition USB port 24 provides means to update software on the device 10. Alternatively software updates can be done wirelessly using Wi-Fi, Bluetooth or cellular communication.

The membrane keypad 18, located below display 16 allows a user to interact with a user interface of the device 10 allowing one to select menu items, change device settings and view pertinent information. As an alternative to membrane keypad 18, the device can instead have a joystick directional input device to allow one to interact with the user interface of the device.

The device 10 also has a toggle on-off switch 28 for placing the device in its off position for long term storage.

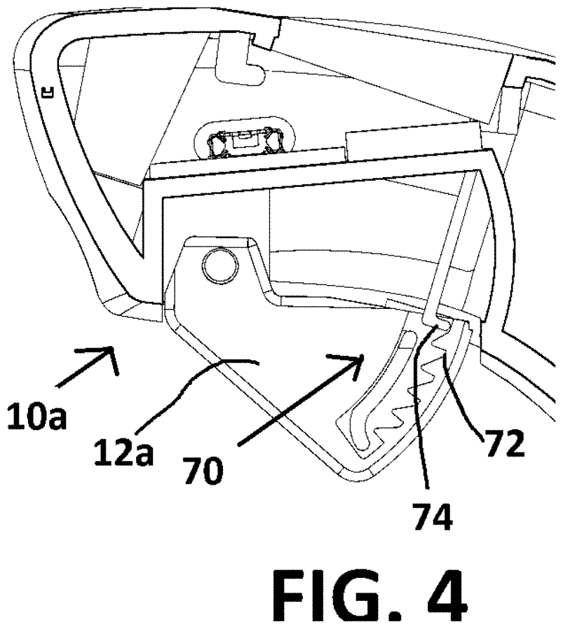

A specific modified embodiment of device 10 is present in device t0a of FIG. 4 in which trigger 12a has been modified from the trigger 12 of device 10 to have a “locking” feature that locks the trigger 12a in place. FIG. 4 is a partial sectional view of this modification and other aspects of device 10a are the same as that of the device 10 (FIGS. 1-3), except as described herein, including aspects and functions and recording contraction data via the trigger 12,12a. The trigger 12a can have a ratcheting mechanism 70 to incrementally lock into place as the trigger 12a travels from fully extended to fully depressed. It can be three clicks, or it can be ten, as two non-limiting examples.

The ratcheting mechanism 70 includes detents or notches 72 on the trigger 12a, that allow the trigger 12a to lock in a fully depressed position by arm 74 engaging the notches 72. The feature permits a laboring woman to convey the intensity of the contraction in an “analog” quantitate way, but relieve her from having to “focus” on holding the trigger in a specific spot. To release the ratcheting mechanism, the trigger 12a will either have to be fully depressed (similar to locking cabinet doors), or optionally have a separate release button like a magazine release on a firearm.

Referring to both devices 10,10a, both can be configured to record contractions as binary data , e.g., depressing the trigger 12, 12a is recorded as a contraction without reference to intensity.

A dedicated SOS button 30 allows the user to press in if one needs immediate attention or to alter one of an emergency. Two or more LEDs 32,34 on the front face of device 10 are for showing various status in accordance with this disclosure.

Additional internal components of device 10 include the main motherboard 20 such as PCBA containing a microcontroller 20a, a wireless baseband modem 20b, antenna 20c, and connectors 20d for other peripherals. A daughter PCBA 21 is present to monitor the trigger 12. The main board 20 also consists of a real time clock (RTC) to maintain an accurate date and time which is maintained by a primary, non-rechargeable battery. An axially polarized magnet 50 is positioned on top of the trigger 12 out of sight of the user.

Advantageously, the device 10 can be constructed to be waterproof in which, within the sealed internal compartment of the device, separate from the trigger compartment 12, is one or more Hall Effect sensors 26 that detect the strength of the magnetic field and can effectively detect presence and/or distance. The analog output of the sensor is fed into an analog input of the microcontroller 20a or processing circuit of the main circuit board 20.

Software running on the microcontroller monitors the analog value and when a pre-programmed threshold is met and debounced, this will be considered as the onset of a user starting to experience a contraction. The microcontroller 20a will instantiate a new object in memory to store information related to the event. The first relevant piece of information is the starting date and time. The microcontroller will communicate to the RTC to get exact time and date.

During an active event, the microcontroller 20a will save the analog value at a periodic interval and save the data to the object as “intensity” values. When the analog value goes below the set threshold, the contraction will be considered over and the date and time will be read from the RTC and stored in memory. The microcontroller 20a will perform the arithmetic to determine the duration and time since last contraction. The display controller will appropriately display all events and statistics on the screen.

When an active Bluetooth LE connection is preset or Wi-Fi, additional features can be achieved by having the event data transferred live (or after event is finished), for example to the user's smartphone running the companion application. The device 10 can generate a checksum of pertinent timing data being transferred to the smartphone app to ensure integrity The companion application can utilize the data connection (Mobile, Wi-Fi or cellular) to upload pertinent data to a remote database. The remote database can be a cloud service or a remote server, e.g., one associated with a healthcare facility, doctor or hospital.

Unique features to the device 10 over prior methods, techniques and devices include the elimination of the inconvenience of having to do conventional “contraction timing” using a watch/clock followed by manually conveying the information to caregivers, a process which is inconvenient and can be prone to errors and the transmission of missing information or absent or missing/unrecorded contractions.

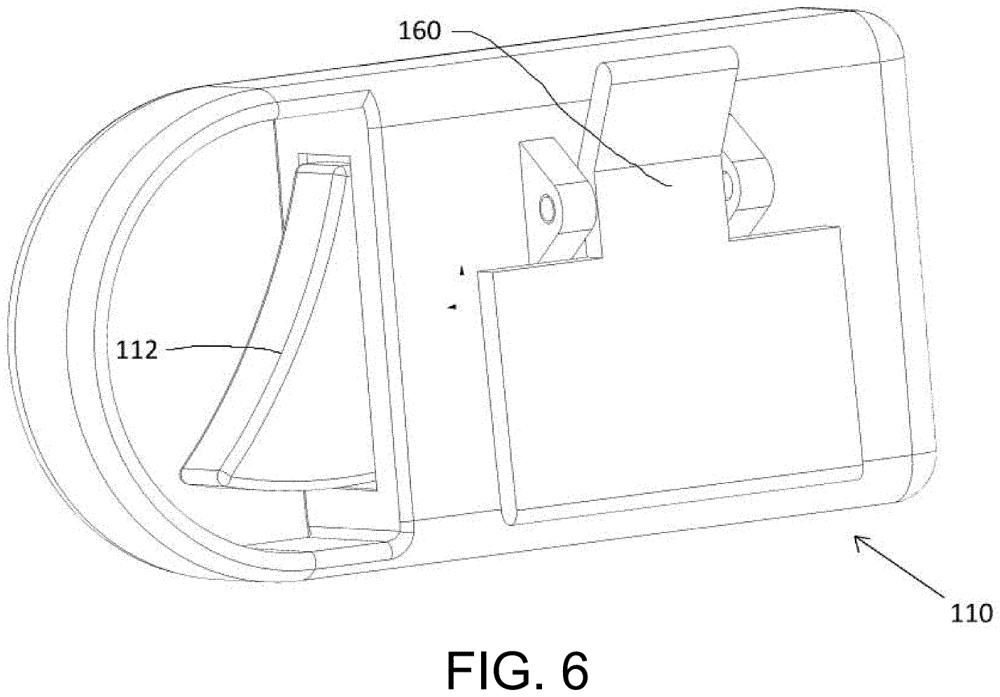

Referring now to FIGS. 5 and 6, device 110 has similar internal components to that of the device 10 for recording contraction data by pressing trigger 112. Accordingly, the device 110 records contraction information by depressing trigger 112 as described above with regard to device 10. As with the alternative device 10a, an alternative embodiment of device 110 can have a modified trigger with a ratcheting mechanism similar to ratcheting mechanism 70 of device 10a.

Distinguishing aspects of device 110 include its output information displayed as duration (DUR), interval (INTV), LAST, 5 AVG, and ALL.

Device 110 replaces the LCD with LED bar graph which provides the user another way to check the pace of their contractions. Depending on which pushbutton is pressed, the bar graph LED lights up the duration and interval of the last contraction, the average of the last five contractions, or all recorded contractions.

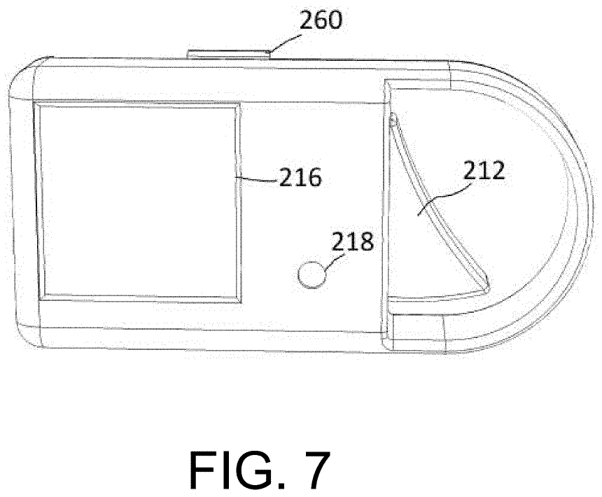

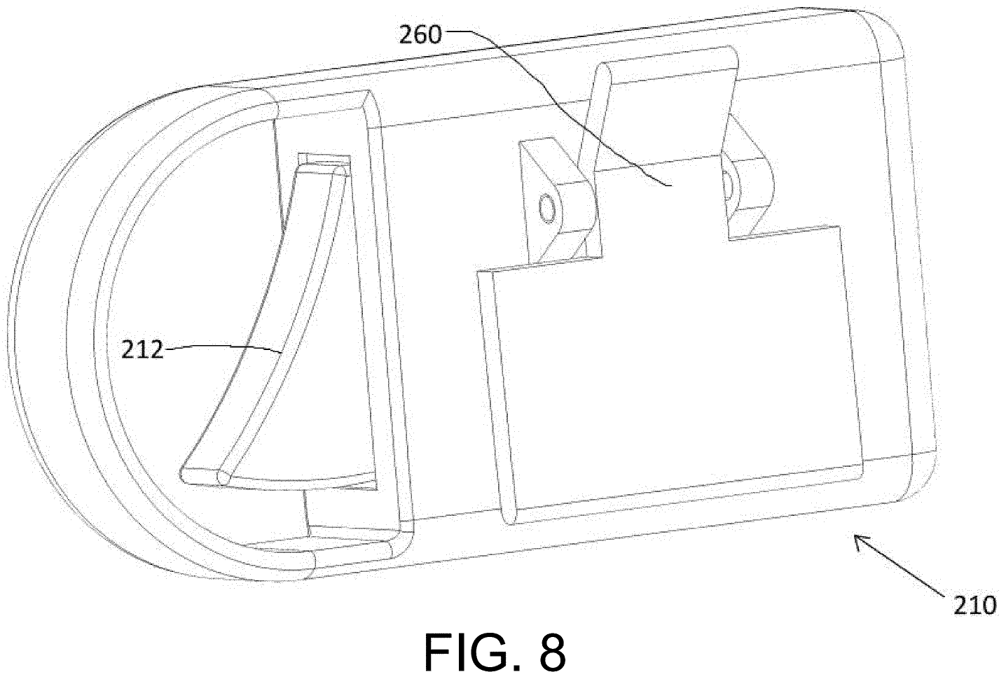

Referring now to the embodiment of FIGS. 7 and 8, a device 210 has internal components essentially the same or functional equivalents of those in device 10 and therefore only differences in device 210 relative to device 10 are described. As with the alternative device 10a, an alternative embodiment of device 210 can have a modified trigger with a ratcheting mechanism similar to ratcheting mechanism 70 of device 10a.

Device 210 has display 216 that provides the information as discussed above with regard to display 16 of device 10. In addition like device 10, device 210 has joystick 218 for manipulating the user interface and selecting various menu items and selecting default or other settings and parameters.

Referring to both devices 110 and 210, clip 160,260 (FIGS. 6 and 8, respectively) allow one to clip the device 110,210 to an appropriate or suitable material or other device.

The present device 10, 110, 210 also is a significant improvement over more modern smartphone conventional contraction timing apps that require fiddling with ones smartphone during the difficulties of labor.

In addition unique aspects of the present device and method include the ergonomic design of the device for use, its wireless including Bluetooth, Wi-Fi, or cellular connectivity, its display or user interface, and conveyance of contraction intensity by means of an analog trigger (non-discrete).

The present device and method can be adapted and modified as necessary or desired to produce a more or less sophisticated system. For example, instead of connectivity to a smartphone or other network device, the method and system can be adapted for use in which the device is standalone and not connected to another device or network in which contraction timing event statistics are displayed on the device's screen.

In an alternative form, the device may not have an analog trigger and Hall Effect sensor. For example, if the analog trigger is replaced with a discrete switch instead, the device can record contractions and prove to be beneficial, convenient and useful to a user.

In yet other alternative forms, various components may be removed (or eliminated) from the present device and the device can take advantage of a cloud-based service platform using a smartphone app to start and stop contraction timing events.

Further, the device may be modified to eliminate the joystick to change the user interface (UI) menu options.

Still further, the external USB port may be eliminated in which the device can still record contractions.

Further alternatives include but are not limited to the following:
Different form factor
   No effect of primary use
No display
   Reduced convenience due to having to open phone app to see statistics, but primary use not effected
Discrete trigger (toggle on/off) switch or button
   Reduced statistical advantage but no effect to primary use
Use of physiological uterine signals rather than depending on user to initiate the start and end via trigger or switch
UI joystick replaced with membrane push buttons, or capacitive touch
   No effect of primary use
LCD replaced with e-ink screen
   No effect of primary use
   E-ink would conserve power and allow for sustained contraction timing data to be present on the screen permanently for quick reference.
Waterproof housing of device

In yet further alternatives, the following modifications may be made:
The status LEDs could be eliminated
The joystick/keypad could be eliminated and combined with the display in the form of a resistive or capacitive touch panel
The on/off switch could be removed
The lanyard loop could be removed and/or changed to a receptacle
The spring providing resistance for the trigger could be eliminated and the “resistive” element could be molded into the trigger (like a diving board)
The separate circuit boards can be combined
The shell securing method could be changed
The o-ring groove could be eliminated and another sealing method implemented
The device can be waterproof

It will now be clear to a person of ordinary skill in the art that further modifications may be made to the present method and system as desired. Alternatives include but are not limited to the following:
SOS button
Attentive or different screens such as a larger screen, smaller screen, or other means of indication such as LED bar graph or LED blink rates, LED color, etc.
Detents on trigger to reduce finger strength required to hold a certain position
Integrated hook instead of lanyard loop hole
Integrated or removable trigger guard
Speaker to confirm trigger pull has been detected
Speaker to provide audible ticks to indicate how far along a woman is in a contraction
Speaker to play relaxation tones, words of encouragement, helpful tips
Lockout switch for children
Trigger can be locked into fully depressed position
Trigger can be a circular loop instead of flat surface (compliments the ability to lock trigger into fully depressed position and then be able to back it away)
Pulse/oximeter sensor on the surface of the trigger to get mothers pulse
Integrated microphone to send voice messages to doctor/caregiver
Message inbox to display messages from caregiver or loved ones

Advantages of the present device and method include wireless connectivity, comfortable form factor convenience and accuracy. This method and system allow a woman to relax during contractions which is important. Midwives and doctors have immediate insights into the rate of progress and can make better judgements as to when to transfer to the hospital or have the midwife visit the patient.

An additional alternative implementation to those of FIGS. 1-8 (i.e., handheld or pendent forms, an variant includes incorporating the functionality of the devices of FIGS. 1-8, into a wrist worn device, e.g. a device attached to a wrist band and to be worn on the wrist.

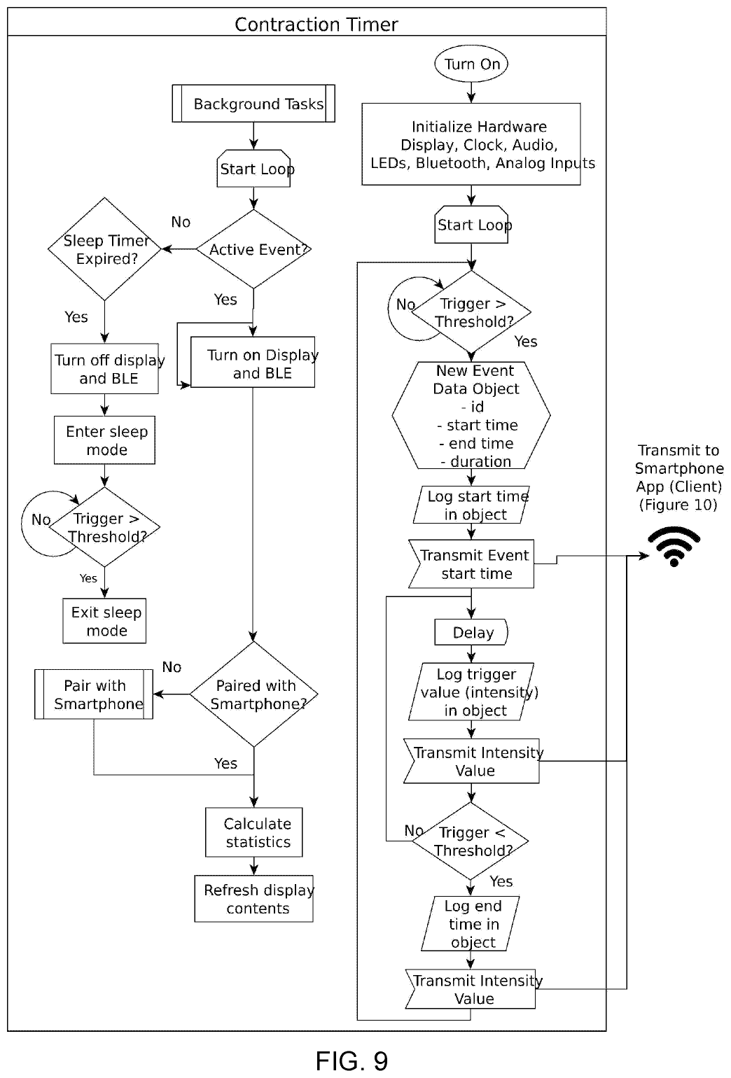

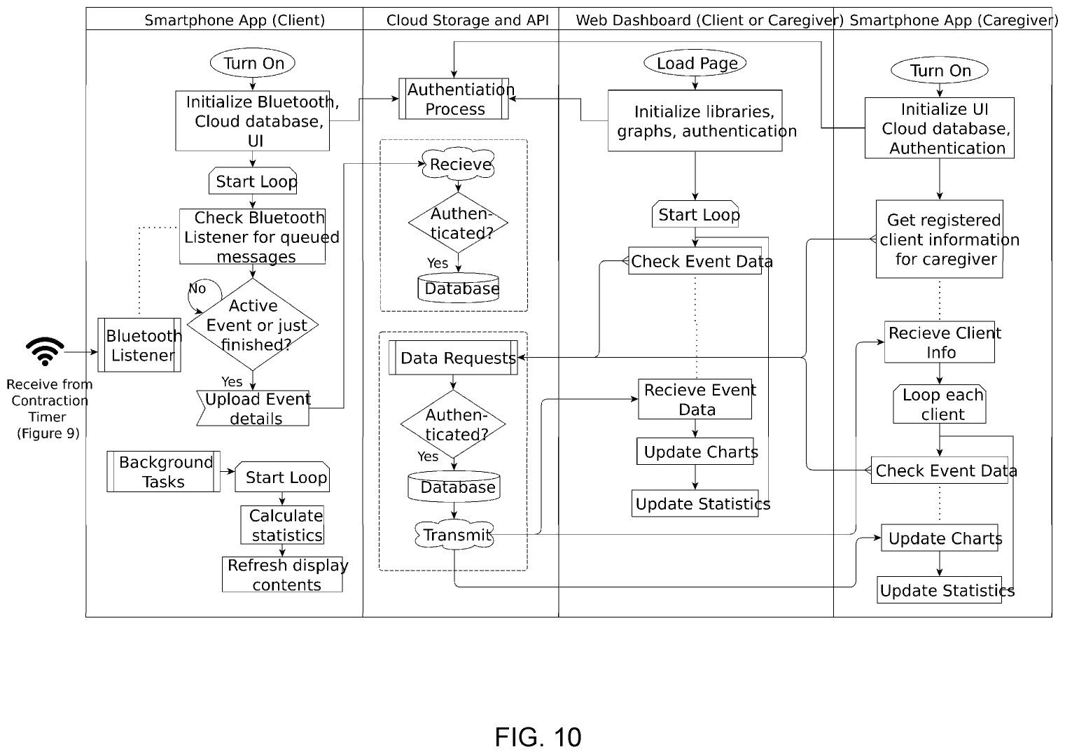

Finally, an exemplary flowchart of the present system is provided in FIG. 9. The flowchart is for a complete system in accordance with the present disclosure. The system shown in FIG. 9 is a non-exhaustive list of components. These components divided by swim lane include:
1. Contraction timer
2. Smartphone App (client)
3. Cloud Storage and API
4. Web Dashboard (client or caregiver)
5. Smartphone App (caregiver)

### Contraction Timer

This swim lane outlines the internal processes taking place within the handheld contraction timer. Upon power up, hardware initialization will take place. Several loops will be active to monitor the analog values of the trigger and to decide if it has crossed any thresholds. If the trigger crosses the pre-defined threshold, then a new event data object is instantiated to record the start time, and ultimately the end time, calculated duration, and any other pertinent metadata. During an active contraction, the intensity values will be transmitted to the Smartphone App as well. Other background processes include maintaining the wireless connection, calculating statistics, and power management.

### Smartphone App (Client)

This swim lane outlines minimum tasks required to receive data from the Contraction Timer. Upon startup, initialization tasks will take place including cloud database setup and authentication, and wireless pairing. The Bluetooth Listener will receive any data wireless data from the Contraction Timer, and process it as necessary, including storing the information in a local database, and cloud database.

### Cloud Storage and API

This swim lane outlines some minimal functions required to authenticate, store, and process requests for contraction timer data. The smartphone apps and web dashboards are the primary sources and consumers of this data.

### Web Dashboard (Client or Caregiver)

This swim lane outlines some minimal tasks to provide a web page. Upon loading of the webpage, software initialization and data source authentication will take place, including processing user login. Data sources are monitored continuously for new information and the information is rendered on the web page when required.

### Smartphone App (Caregiver)

This swim lane outlines minimum tasks required for the caregiver to receive data about their patients (aka client). Upon startup, initialization tasks will take place including cloud database setup, authentication, and determination of registered patients. For each client, the app will monitor for new information and will update the screen with information as needed.

### Web Dashboard (Client or Caregiver)

The present system has a secure online portal for displaying contractions and statistics. This gives mothers, caregivers, and their loved ones a simpler way of tracking the progress of labor, and perform interactions.

While the flowchart (FIG. 9) is a complete system, a person of ordinary skill in the art will understand that numerous modifications may be made to the system, adding and/or eliminating aspects or elements, consistent with the present disclosure.

### Glossary of Alternative or Equivalent Terms

Trigger→Sensor, Analog-to-digital converter
Spring-loaded→Spring-biased
Hall effect→Magnetic sensor
Detents→Notches
RTC→Timer
Circuit board→Processing circuit

Although the invention has been described with respect to a preferred embodiment, it is to be understood that the invention is capable of numerous modifications and variations, apparent to those skilled in the art, without departing from the spirit and scope of the invention.

1. A device for monitoring the childbirth and the laboring process, said device comprising:
a housing with a trigger, the trigger being force sensitive, wherein more force applied to the trigger is indicative of contraction intensity.

2. The device of claim 1, further comprising recording contraction data generated when force is applied to the trigger.

3. The device of claim 1, further comprising a wireless communication system for transmitting contraction data generated when force is applied to the trigger.

4. The device of claim 3, wherein the wireless communication system is selected from the group consisting of Bluetooth, Wi-Fi or cellular.

5. The device of claim 1, further comprising a display for displaying contraction data generate as a result of depressing the trigger and force applied to the trigger.

6. The device of claim 4, where in the wireless communication system is connected to a remote device.

7. The device of claim 6, wherein the remote device is selected from the group consisting of a smart phone, cloud server, and a remote server.

8. The device of claim 1, wherein the trigger has a locking feature that locks the trigger in place.

9. The device of claim 8, wherein the locking feature comprises a ratcheting mechanism that incrementally locks the trigger in place as the trigger is depressed from a fully extended position to a fully depressed position.

10. The device of claim 1, wherein the trigger further comprises a damper to soften a return of the trigger after the trigger has been pressed, from a partial or a fully depressed position to its quiescent position.

11. An ambulatory device for measuring the frequency and duration of uterine contractions, the ambulatory device comprising:
a processing circuit;
an analog-to-digital converter coupled to the processing circuit;
a biased trigger sensor configured to receive a manually applied time-varying input force signal over a monitoring period, the trigger sensor being coupled to the analog-to-digital converter; and
a wireless transceiver coupled to the processor;
wherein the trigger sensor outputs a time-varying analog signal to the analog-to-digital converter, the analog signal being monotonically increasing with an increasing manually applied input force signal and representative of the manually applied input force signal over the monitoring period; and
wherein the processing circuitry transmits a digital output signal via the wireless transceiver, the digital output signal being indicative of the manually applied input force over the monitoring period.

12. The ambulatory device of claim 11, wherein the biased trigger sensor includes:
a trigger; and
a spring.

13. The ambulatory device of claim 12, further comprising a detent positioned to detain the trigger when the trigger is displaced beyond a first displacement threshold.

14. The ambulatory device of claim 12, further wherein the trigger has a locking feature that locks the trigger in place.

15. The device of claim 14, wherein the locking feature comprises a ratcheting mechanism that incrementally locks the trigger in place as the trigger is depressed from a fully extended position to a fully depressed position.

16. The device of claim 12, wherein the trigger further comprises a damper to soften a return of the trigger after the trigger has been pressed, from a partial or a fully depressed position to its quiescent position.

17. The ambulatory device of claim 11, wherein the wireless transceiver is adapted to transmit contraction data generated when pressure is applied to the trigger senor.

18. The ambulatory device of claim 14, wherein the wireless transceiver is selected from the group consisting of Bluetooth and Wi-Fi and cellular.

19. The ambulatory device of claim 11, further comprising a display for displaying contraction data generate as a result of depressing the trigger and pressure applied to the trigger.

20. The ambulatory device of claim 11, where in the wireless transceiver allows a wireless connection to a remote device selected from the group consisting of a smartphone running an app, cloud server/database and a healthcare facility server.
